(19) United States
(12) Patent Application Publication
(10) Pub. No.: US 2007/0184011 A1
(43) Pub. Date: Aug. 9, 2007

(54) SHAVING GEL COMBINATIONS AND DEVICES

(76) Inventor: Marc S Herlands, Encinitas, CA (US)

Correspondence Address: WESLEY B. AMES, 7031 LOS VIENTOS SERENOS, ESCONDIDO, CA, 92029, US

(21) Appl. No.: 11/530,775

(22) Filed: Sep. 11, 2006

Related U.S. Application Data
(63) Continuation-in-part of application No. 11/346,887, filed on Feb. 3, 2006.

(51) Int. Cl. A61K 8/36

(52) U.S. Cl. 424/73

(57) ABSTRACT

A shaving composition is described that combines a shaving gel, foam, or cream, and a liquid hand or bath soap, along with razors having improved cleaning characteristics and designs, along with methods for using such shaving compositions and razors.

### RELATED APPLICATIONS

This application is a continuation-in-part of Herlands, U.S. patent application Ser. No. 11/346,887, filed Feb. 3, 2006, which is incorporated herein by reference in its entirety.

### FIELD OF THE INVENTION

The present invention relates to shaving products and methods of using those products, and especially to shaving softening and lubricant compositions and particular types of razors.

### BACKGROUND OF THE INVENTION

The following discussion is provided solely to assist the understanding of the reader, and does not constitute an admission that any of the information discussed or references cited constitute prior art to the present invention.

A large variety of different compositions have been used for shaving purposes, ranging from solid shaving soaps that can generate a lather, to shaving creams, shaving foams, and to post-foaming shaving gels. Similarly, a number of liquid soap compositions have been described. A few examples of patents describing liquid soaps and shaving compositions are indicated below.

MacGilp, U.S. Pat. No. 5,296,158 describes a “mild, stable, liquid disersoidal cleansing composition comprising 55% to 90% water; 5% to 20% saturated (low IV) higher (high titer) fatty acid potassium soap; 2.5% to 18% of free fatty acids”, with specified ratios of soap and free fatty acids and with a viscosity of about 4000 cps to about 100,000 cps at about 25 degrees C.

Helioff et al., U.S. Pat. No. 5,034,220 describes a non-aerosol shaving gel which “consists essentially of a resin gel-former which is a crosslinked, neutralized copolymer of maleic anhydride and a C₁-C₅ alkyl vinyl ether, a water-soluble soap and water.”

Hartmann et al., U.S. Pat. No. 5,902,778 describes a post-foaming gel shaving composition “which contains from about 40 to about 90 percent by weight water; from about 4 to about 25 percent by weight water-soluble soap; from about 0.5 to about 12 percent by weight aliphatic liquid post-foaming agent selected from the group consisting of saturated aliphatic hydrocarbons; from about 0.01 to about 5 percent by weight of at least one water-soluble gelling agent forming in said composition a gel having a yield value sufficiently high to restrain said composition from foaming for at least about 60 seconds; and from about 0.1 to 2 percent by weight of a poly(ethylene oxide) having an average molecular weight of at least 2×10⁵.”

A large number of patents describe razors and razor blade units, including some having two shaving heads.

For example, Sceberras, U.S. Pat. No. 4,501,066 describes “a dual headed razor systems having a handle supporting a pair of separately detachable razor heads respectively useful in shaving forwardly and rearwardly in to and fro strokes. The identical razor heads are usable interchangeably and are telescopically supported crosswise of the handle.”

Eltis, U.S. Pat. No. 4,831,731 describes a multi-blade safety razor which includes “a shaving head and a handle that extends therefrom. The shaving head includes at least two pairs of dual razor blades, each blade having a single shaving edge. The dual blade arrangements are fixedly mounted on the shaving head in a substantially longitudinal manner in order that the shaving edges of all blades are exposed in a common plane. A skin engagement surface is provided between each dual blade arrangement for providing a closer shave.”

Hoffman, U.S. Pat. No. 5,133,131 describes a replaceable, double-sided razor blade unit for use with a handle member in a shaving system.

Ahlgren, U.S. Des. 343,922 shows a dual headed disposable razor with oppositely directed blades.

Mondo et al., U.S. Pat. No. 5,287,624 describes a disposable safety razor which has “a head having a convex top surface and a flat bottom surface attached to an upper end of the elongated handle. A pair of single cutting edge razor blades are clamped parallel within the head with the cutting edges angled outwardly and upwardly in opposite direction from the convex top surface of the head, so as to be useful in shaving in both forward and rearward strokes.”

Andrews, U.S. Pat. No. 5,343,622 describes a bi-directional razor device which “is formed of a narrow elongated head and a transversely extending, integral hand grip formed of a molded plastic material. Two pair of narrow, strip-like razor blades are embedded in the head, with one pair extending in one direction and the other pair extending in an opposite direction at an acute angel relative to the first pair.”

McNinch, U.S. Pat. No. 5,426,853 describes an improved shaver having a bifurcated body; an adjustable handle attached to the center of said body; said adjustable handle and said body forming a Y-shape; and at least two holders, each of said holders being sized and shaped for holding razor blades attached to the ends of the body by rotators.”

Andrews, U.S. Pat. No. 6,434,828 describes an in-line razor device with twin pivoting heads, which “feature two sets of razor blade strips pointing inwardly in opposite directions.”

Wexler, U.S. Pat. No. 5,630,275 describes a multi-blade razor head with improved performance “wherein the physical characteristics of the cutting edges are different from each other. Among the items which may be varied in the blade are the substrate material, coatings or shape.”

### SUMMARY OF THE INVENTION

This invention concerns advantageous methods and compositions for shaving. Applicant has found that adding a substantial proportion of a liquid hand or bath soap to a shaving gel provides improved lubrication and significantly longer blade life, and also that quite advantageous efficiencies are obtained by using a multi-stage (e.g., two stage) shaving method. Such multi-stage shaving method can utilize multiple blade units, which can, for example, be with single-headed or multi-headed razors (such as two-head razors) using blade units with different numbers of razor blade strips.

Thus, in a first aspect, the invention provides a shaving composition that combines a shaving gel, foam, or cream and a liquid hand or bath soap. For example, the composition can include 20-80% by weight of a shaving gel (such as a post-foaming shaving gel), foam, or cream, and 20-80% by weight of a liquid hand or bath soap.

In particular embodiments the shaving composition includes 40-60% of the shaving gel, foam, or cream, and 40-60% of the liquid hand soap, or 45-55% of the shaving gel, foam, or cream, and 45-55% of the liquid hand soap.

The invention also concerns a shaving composition dispenser that contains a shaving gel, foam, or cream and a liquid soap in separate chambers, such that the dispenser will jointly dispense the shaving gel, foam, or cream and the liquid soap. Thus, another aspect provides a shaving composition dispenser that includes two chambers, where the first chamber contains a shaving gel, foam, or cream, the second chamber contains a liquid hand soap, and the dispenser dispenses a pre-selected ratio of each of the liquid hand soap and the shaving gel, foam, or cream.

In particular embodiments, the shaving gel, foam, or cream and the soap are dispensed from pressurized chambers; the shaving gel, foam, or cream and the soap are dispensed from separate pressurized chambers; the shaving gel, foam, or cream and the soap are dispensed from separate pressurized chambers and dispensed from a common aperture; the shaving gel, foam, or cream and the soap are dispensed from separate pressurized chambers and are dispensed from separate apertures.

A related aspect concerns a shaving composition dispenser that includes two linked containers, one of which contains a shaving gel, shaving foam, or shaving cream, and the other of which contains a liquid soap, typically a liquid hand or bath soap. Such linkage can, for example, involve a single outer container wall with a single internal barrier wall, separate containers that are joined by welding, or separate containers that are joined with a plastic or metal band. Each container has a separate actuator, usually but not necessarily leading to a separate outlet.

In yet another aspect, the invention concerns a shaving method that involves applying both a shaving gel, foam, or cream and a liquid hand or bath soap to a skin surface to be shaved, and shaving that skin surface. The shaving gel, foam, or cream and the liquid hand or bath soap can be used as a combination of the two materials. For example, the method can include applying a combination of one part by weight of a shaving gel, foam, or cream and 0.2-5.0 parts by weight of a liquid hand soap to a skin surface to be shaved, and shaving that skin surface. Alternatively, the soap and the shaving gel, foam, or cream can be applied sequentially, e.g., first applying the soap and then, either with or without removal, applying the shaving gel, foam, or cream followed by shaving. In other cases, the gel, foam, or cream is applied first and then, either with or without removal, the liquid soap is applied and the area is then shaved. The second material is applied essentially immediately, or at least before the first applied material and/or the hair on the area to be shaved has dried.

In particular embodiments, the combination is as described for an above aspect.

As indicated above, the invention concerns a multi-stage, e.g., at least two-stage, wet shaving method. The first stage provides a less than desired shave closeness by using a razor having a blade unit which has one or more (e.g., 1, 2, or 3) blade strips, while the second stage provides a closer shave by using a shaver having a blade unit which has at least one additional blade strip compared to the blade unit used in the first stage, e.g., a blade unit having three-, four- or five-blade strips.

In particular embodiments, two different razors are used; a dual-head razor is used; a razor is used which mounts two blade units in sequential orientation is used; a razor is used in which two blade units are mounted in sequential orientation arranged such that, beginning from an orientation in which the first blade unit is in proper orientation for shaving in stage one, changing the angle of the razor handle to a steeper angle relative to the user's skin brings the second blade unit into contact orientation in stage two; a triple head razor is used.

In certain embodiments, the first blade unit includes only one blade strip and the second blade unit has at least 2 blade strips; the first blade unit has two blade strips and the second blade unit has at least 3 blade strips; the first blade unit includes a single blade strip and the second blade unit includes 3, 4, or 5 blade strips; the first blade unit has 2 blade strips and the second blade unit has 3, 4, or 5-blade strips, the first blade unit includes 3 blade strips and second blade unit has 4 or 5 blade strips; the first blade unit has 4 blade strips and the second blade unit has 5 blade strips.

This shaving method can utilize the liquid soap and shaving gel, form, or cream as otherwise described above or otherwise described herein.

In a related aspect, the invention concerns a shaving kit which includes blade units of at least two different shaving closeness levels and/or different cost levels packaged together. In a kit containing two types of blade units, one blade unit will have at least one blade strip and the second blade unit will have at least two blade strips, that is, the second blade unit will have at least one more blade strip than the first blade unit. Many different combinations of blade units may be provided in such kits which use different numbers of blade unit types with blade units having different numbers of blade strips. For example, a first blade unit type may be 1- or 2-blade strip blade units, while a second blade unit type may be 3-, 4-, or 5-blade strip blade units, or the first blade unit may be a 3-blade strip blade unit and a second blade unit may be a 4- or 5-blade strip blade unit. It is expected that in most cases, two types of blade units will be included, but, if desired kits may include additional blade unit types, e.g., 3 or 4 different blade unit types.

In particular embodiments, the kit also includes a razor handle and/or instructions for using the substantially different blade units jointly in a shaving process, e.g., the shaving method indicated above. Blade units and associated razors as described for the preceding shaving method may be included in the present shaving kits.

Instructions included in the kit may comprise written directions for using the at least two different types of blade units and/or for performing shaving using both liquid soap and shaving gel, foam, or cream, e.g., a method as indicated above using either a combination of the liquid soap and shaving gel, foam, or cream, or using those materials sequentially.

A related aspect concerns a multi-head, e.g., dual-head, razor which comprises at least two different blade units such that blade units on the razor respectively have at least two different numbers of blade strips, e.g., combinations as indicated for the kits described above.

In particular embodiments, multiple heads (e.g., dual heads) are configured such that blade units mounted on the respective heads are in the same plane; the blade units are in different planes; the blade units are arranged such that, beginning from an orientation in which the first blade unit is in proper orientation for shaving, changing the angle of the razor handle to a steeper angle relative to the user's skin brings the second blade unit into contact orientation; the blade units are on opposite sides of the axis of the razor handle and the edges of the blade strips of the two blade units are oriented away from that axis.

Another aspect concerns a razor blade unit (and/or a razor having such blade unit) which includes at least three blade strips having decreasing spacing, e.g., with the spacing between the first and second blade strips being greater than between the next-to-last and last blade strips. Such arrangement can provide greater capacity for shaving debris to clear the blade strips and/or provide improved clearing characteristics.

In particular embodiments, the debris throat before the first blade strip is at least 30, 40, 50, 70, or 100% wider than the debris throat between the next to last and last blade strips; the debris throat between the first and second blade strips is at least 20, 30, 40, or 50% greater than the debris throat between last and next to last blade strips; the width of the debris throat before the first blade strip is at least 1.3, 1.5, 1.7, 2.0, 2.5, or 3.0 times the width of the debris throat between the most closely spaced pair of blade strips in the blade unit.

In certain embodiments, the razor includes a clearing plunger operable using contact on the razor handle or head by which a user can cause the clearing plunger to extend into and/or through a least one debris throat, e.g., the first and/or second debris throat.

The term “razor” is used conventionally to refer to a shaving device, generally for wet shaving, which includes a handle and a blade unit which has one or more generally straight blade strips. In many cases, the blade unit is readily replaceable on the head of the razor handle.

As used herein, the terms “razor blade”, “blade unit”, and “blade assembly” refer to a disposable assembly for shaving, which includes one or more cutting edges or blade strips. Thus, the term “blade strip” refers to a cutting blade (also referred to as a cutting edge) in a razor blade unit or assembly, e.g., a disposable blade unit.

The term “debris throat” is used to refer to the gap preceding a blade strip into and through which shaved hairs, along with shaving cream or gel are forced during shaving. For multi-blade razor blade units, there will be a debris throat preceding the first blade strip and between each pair of adjacent blades strips.

In connection with mechanisms for cleaning or clearing debris from between blade strips in a blade unit, the term “plunger” refers to a structure in a razor handle or a handle and/or a blade unit which can be extended or thrust into or even through the debris throat between two blade strips (or into two or more such debris throats).

As used in connection with the present invention, the phrases “shaving blade unit”, “blade unit”, “razor blade unit”, and “razor blade” refer to components of a razor which contain at least one cutting edge and which are designed and intended for shaving facial and/or other body hair, as distinguished from other hair cutting techniques such as trimming (e.g., trimming moustaches, beards, and sideburns). Thus, the phrases “different blade unit type”, “different blade unit”, and like phrases refer to differently constructed blade units designed and intended for shaving, e.g., blade units which include different numbers of blade strips. While the present razors may optionally include trimming blades, such trimming blades shall not be deemed to be “blade units” as that term is used herein.

Reference to “different shaving closeness levels” in connection with different razor blade units means that the average shaving closeness resulting from use of new blade units of the respective types results in differences in shaving closeness noticeable to users of normal perceptiveness.

Reference to “different cost levels” in connection with different razor blade units means that for the referenced blade units, the average non-discounted prices per blade unit of the referenced blade units differ in at least a market area relevant for a purchaser, e.g., the price of the more expensive blade unit is at least 1.1, 1.2, 1.3, 1.5, 1.7, 2.0, 2.5, or 3.0 times the price of the less expensive blade unit in that market.

Additional embodiments will be apparent from the Detailed Description and from the claims.

### BRIEF DESCRIPTION OF THE DRAWINGS

FIG. 1 shows an exemplary dual dispenser for a shaving gel, foam, or cream and liquid soap combination.

FIG. 2 shows an exemplary dual dispenser for a shaving gel, foam, or cream and liquid hand soap combination that has two separate chambers and separate actuators and dispensing apertures.

FIG. 3 shows a two-head razor suitable for the present two-stage shaving method.

FIG. 4 shows a dual head razor having sequentially arranged mounting of two different razor blade units.

FIG. 5 shows a cross-section of a disposable multi-blade unit having improved cleaning characteristics.

FIG. 6 shows an exemplary plunger mechanism for improved cleaning for a multi-blade shaver.

### DETAILED DESCRIPTION OF THE PREFERRED EMBODIMENTS

The present invention concerns advantageous compositions and methods for wet shaving, including hair softeners and lubricants and improved disposable blade shavers.

### Shaving Gel and Hand or Bath Soap Combinations

Such hair softeners and lubricants concern combinations of shaving gel (or shaving foam or cream) and hand or bath soap. The combination remains on the skin, providing softening of hair and lubrication. In many cases, the combinations can also provide extended useful life for razor blade units.

In certain embodiments, the combination is provided as separate compositions that are jointly dispensed and mixed for use. Such combinations have the advantage that the foaming properties of the shaving gel or foam are not substantially impaired.

In other embodiments, a new formulation is provided that includes a substantially higher proportion of soap or detergent components than conventional shaving gels. Thus, the formulation can be regarded as a mixture of a shaving gel and a liquid hand or bath soap.

In many cases, shaving gels contain a combination of water, soap, gelling component(s), and post-foaming gas, along with additional components such as fragrances, emollients, and/or glidants.

A number of shaving gels, especially post-foaming gels, that can be used have been described. For example, U.S. Pat. Nos. 6,916,468; 6,878,754, 6,353,689; 5,902,778; 5,902,574; 5,858,343; 5,759,531; 5,560,859; 5,523,081; 5,262,154; 5,248,495; 5,034,220; 4,871,530; 4,528,111; 3,541,581, as well as patent application publications 2004/0166086; 2004/0166085; and 2003/0170198 describe such shaving gels.

Likewise a large number of different liquid hand and bath soaps can be used. In many cases, such liquid soaps contain a soap or detergent, along with additional ingredients conventional for such liquid hand soaps.

Examples of liquid hand and bath soaps that can be used have been described. For example, U.S. patents MacGilp et al. U.S. Pat. No. 5,296,158; Shields U.S. Pat. No. 4,617,148; Holsopple U.S. Pat. No. 4,556,510; Beattie U.S. Pat. No. 4,430,245; Matsuda U.S. Pat. No. 4,312,771; Brouwer U.S. Pat. No. 4,310,432 describe such liquid hand and bath soaps.

The present shaving gel/hand soap combinations can be dispensed from a number of different types of containers, e.g., pressurized or pump containers as currently used for shaving gels and/or liquid hand soaps. In the case of combination formulations (i.e., shaving formulations containing components similar to the combined ingredients of current shaving gels and liquid hand soaps, a single chamber dispenser can be used, such as the pressurized dispenser cans typically used for dispensing post-foaming shaving gels. Such dispenser cans commonly incorporate a gas-pressurized bag-in-can or diaphragm design, or a spring-pressurized divider. Such dispensers can include valve assemblies as described and/or commonly used for use with post-foaming gels or with aerosol gels.

The stable post-foaming gel can be packaged in a variety of container systems, preferably containers that include propelling structures such as: (1) mechanically pressurized bag-in-sleeve systems in which a thin-walled inner bag containing the product is surrounded by an outer elastic sleeve that is expanded during the product filling process and provides dispensing power to expel the product (e.g., the ATMOS System available commercially from the Exxel Container Co.); (2) manually activated air pump spray devices in which a pump system is integrated into the container to allow the user to pressurize the container with air in order to expel the product (e.g., the “AIRSPRAY” system available from Airspray International); (3) piston barrier systems (or can with piston) in which the product is separated from the driving means by a tight-fitting piston which seals to the side of the container and may be driven by a spring under tension, by a vacuum on the product side of the piston, by finger pressure, by gas pressure to the piston, or by a variety of other means known to the packaging industry; and (4) bag-in-can (SEPRO) systems in which the product is contained in a flexible bag within a can, with a suitable propellant injected into the space between the can and the flexible bag.

In particular, the “bag-in-can system” basically consists of a metal 3 piece can equipped with an inner plastic bag fastened to the dome that contains the gel/post-foaming agent mixture. The propellant is loaded through the bottom of the can which is perforated for a charging plug, and the propellant remains separate from the bag. The container is designed to use hydrocarbons, and other compressed gasses. The bag serves as a barrier to prevent the gel/post-foaming agent mixture from coming in contact with the propellant around the bag. The bag is made of polyethylene, Surlyn and nylon and has pleated side walls which enable the bag to collapse upward as the propellant force empties the container. The bag is capable of discharging very viscous materials. A major problem with this unit is gas permeation through the bag into the gel. Permeation occurs “when significant numbers of molecules pass through a barrier during a fixed time period.” Larger molecules permeate more slowly than smaller ones. Entrapment of air in the gel when filling into the can will be minimized by use of a spin filler, which rotates the cans while gel is transferred into the bag.

In addition, manually activated pumps can be used. Manually activated pumps typically include a squeeze trigger or finger-type pumping device as part of the propelling structure. Such pumping devices are well known in the art and typically include a pump chamber, an injection pipe member connected to a vertical pipe portion (piston), a cylinder, and a discharge valve, which communicates with the pump actuator. Fluid (or gel) is forced from the pumping device under pumping pressure generated by the pumping action of an injection pipe member lifted and lowered vertically in a cylinder by an operating mechanism. The injection pipe member includes a vertical pipe portion, corresponding to a piston, which is forced into the cylinder. The fluid pressurized in the pump chamber exerts pressure, through means of a fluid passage in the injection pipe member, onto and through a flexible discharge valve, thereby causing the fluid to be ejected from the actuator. A wide variety of manually activated pump dispensers may be used.

In other cases, the dispenser includes separate chambers for the shaving gel component and the liquid hand soap component. In such dispensers, dispensing of each component may be controlled in various ways, and the dispensing channels may be combined or separate. Thus, actuation of the dispenser may involve separate actuation of dispenser controls for each component. In such configurations, each component will typically, but not necessarily, issue from a separate aperture. Such separate apertures may be completely separate or may be twinned. Alternatively, the separate components may utilize the same aperture. In other embodiments, a single control actuates dispensing of both components jointly; the separate components may combine internally and issue through a common aperture, or may issue through separate apertures, e.g., twinned apertures.

Each of the separate chambers may be configured with propelling structures, such as those described above. In some cases it may be preferred to use a positive displacement mechanism such as a piston in can system in conjunction with separate flexible bags within a single container. The bags ensure that the components are maintained separately, while the piston ensures that a volume (e.g., an aliquot) of each component is dispensed. This will prevent one component (e.g., a less viscous component) from being dispensed in preference to the other component. It is generally preferable that the components be dispensed in a substantially consistent ratio (e.g., within a factor of two or even 1.7, 1.5, 1.4, 1.3, or 1.2); thus, the dispensing mechanism be configured to provide such substantially consistent ratio.

The combination of shaving gel, foam, or cream, and liquid hand or bath soap can also be provided in separate containers that are linked or packaged together. In such cases, dispensing the two items will generally occur through separate valve mechanisms and dispensing orifices. Such linkage or packaging can, for example, involve binding of the two containers together with a plastic or metal band(s), gluing together of the containers with or without a pack or frame, or the containers may fit within openings in a pack or frame that holds the containers without the requirement for glue or other adhesive.

Dispensers can be used that will dispense either a single aliquot (or an aliquot of each component) on each actuation, or will dispense essentially continuously while an actuator is held in an actuating position. Valve assemblies of both types are well known and can be used for the present dispensers.

The liquid soap and shaving gel/cream combinations can be used in advantageous shaving methods. In addition to using combinations or blends of the two components, the liquid soap and shaving gel/cream can be used sequentially. Thus, the skin surface to be shaved can be washed using the liquid soap and the soap removed by rinsing or wiping. Shaving gel or cream can then be applied and the area shaved.

### Razors Having Improved Cleaning Characteristics and/or More Economical Blade Unit Usage

The improved razors indicated above include several different variations of multi-blade razor blade units which improve the user's ability to clean the blade units of shaving debris and/or provide a more economical shaving practice while retaining the close-shave benefits of blade units having multiple blade strips, e.g., 3 or more blades.

In particular, razors can be constructed (and can be provided alone or in shaving kits) such that at least two different razor blade units are provided on a single razor handle. The blade unit having the least number of blade strips provides a “rough cut”, but does not provide a desirably close shave. A second, (or second and subsequent) blade unit on the same handle then is used to provide a closer shave than that provided by the “rough cut” blade unit. In most cases, the first blade unit will have one or two blade strips, but in some cases will be a unit with 3 or 4 blade strips. Usually the first blade unit will be substantially cheaper than the second and/or subsequent blade unit. In most cases, a second blade unit (and/or subsequent blade unit) will have at least 3 blade strips, e.g., 3, 4, or 5. Such blade units are usually significantly more expensive than the blade units with 1 or 2 blades, but are typically designed to provide an appreciably closer shave.

Thus, the first blade unit is used first and provides a primary shave (which can be regarded as a “rough cut”), but the blade unit may generally become dull before the second and/or subsequent blade unit. The second blade unit is used second, and provides a closer shave. By following the first blade unit, the second blade unit is not exposed to the same dulling effect as if it were used alone, which may result in substantially longer useable lifetime. Similarly any subsequent blade units, e.g., 3^{rd}, 4^{th}, etc., are also not exposed to the same dulling effect as if they were used alone or as the first blade unit. Because of the typical difference in cost for the 1^{st} and 2^{nd} (and/or subsequent) blade units, the extended lifetime for the 2^{nd} (and/or subsequent) blade unit may result in lower costs per shave as per the individual's general method of shaving a particular portion of the body.

The sequential use of the two or more different types of blade units can involve simultaneously use of a razor which holds the two or more blade units such that the two or more blade units shave essentially continuously. Alternatively, a razor may hold two or more types of blade units, but only one is in contact with the user's skin at a give time, and the razor is repositioned to bring the second blade unit (and similarly any subsequent blade units) into contact with the skin. The latter configuration may be accomplished by having the two or more blade units in different planes. The different planes may have only a small angle between them, e.g., such that rotating the handle about a transverse axis through the shaving head(s) brings a second blade unit into position to contact the user's skin. In such embodiments, the angle between the planes will often be about 5-30 degrees. In an alternative, the angle between the planes is relatively large, e.g., about 60-90 degrees. In such embodiments, the different blade units can be arrayed around the longitudinal axis of the razor handle, e.g., on opposite sides of the longitudinal axis of the handle. Shifting from one of the blade units to the other is normally accomplished by rotating the handle about it longitudinal axis, e.g., about 180 degrees for two blade units, about 120 degrees for 3 blade units, and about 90 degrees for 4 blade units.

In addition to the relatively high cost of blade units having 3 or greater blade strips (e.g., 3, 4, or 5), one of the difficulties of using such blade units is that they readily clog with a mass of cut hairs intermixed with shaving gel, cream, or soap. Such blade units having 3 or more blade strips are typically constructed with an opening or throat in front of each blade strip. In current designs, the throats are relatively narrow and each throat is approximately the same width within a blade unit. In many cases, one or more of the throats become clogged. The clogging is usually worst in front of the 1^{st} blade and in the throat between the 1^{st} and 2^{nd} blade strips. Such clogging can result in difficulties in cleaning the blade unit for continued shaving. Such clogging is exacerbated when the hair being shaved is relatively long and/or heavy. In such case, very vigorous washing may be necessary to clear the debris.

The problem of clogging can be reduced (without substantially reducing the closeness of the shave) by providing larger debris throats, especially for the leading blade strips, e.g., in front of the 1^{st} blade strip and/or between the 1^{st} and 2^{nd} blade strips. With such blade units, the 1^{st} blade strip cuts the majority of the hair, with the wider debris throat accommodating the larger amount of debris. The second blade strip also cuts a substantial amount of hair, so if the debris throat preceding the 2^{nd} blade strip is also increased in size, clogging of this debris throat will also be decreased. The spacing between subsequent blade strips can then be reduced progressively or reduced to a uniform separation. The narrower debris throats for the subsequent blade strips will be less prone to clogging because the debris from cut hair and associated soap, gel, and/or foam will be much reduced for those subsequent blade strips.

Either as an adjunct to the varied blade strip separation, or in association with uniform separation blade strips, cleaning will also be enhanced by providing a clearing plunger. Such plunger is operated via an actuator (e.g., a button or slide) on the razor handle or head. The plunger extends into or through one or more debris throats when actuated, pushing the debris out of the throat and thereby facilitating clearing of the throat. The plunger(s) can, for example, be configured and positioned to clear the 1^{st} debris throat, or the 1^{st} and 2^{nd} debris throats, or the 1^{st}, 2^{nd}, and 3^{rd} debris throats, or all of the debris throats.

The razor blade units having enhanced clearing properties can be used in shaving methods utilizing only one blade unit, or in methods such as those described above in which at least two different types of blade units are used. While the various blade units described can be constructed integral with a razor handle, in most cases it will be advantageous for the blade units to be readily separable from the handles, and in cases in which two different types of blade units are installed, independently replaceable. Thus, preferably, the present razors utilize disposable blade units.

The present razors and razor blade units can be constructed using methods and materials well-known in the field, including methods and materials are indicated or described in patent documents listed in the Background of this description and in patent documents cited in those listed patent documents.

### EXAMPLES

As indicated above, combinations of shaving gel and liquid hand or bath soap that can be used in the present invention are numerous. To illustrate the invention further, an exemplary dual chamber dispenser and an exemplary combination of shaving gel and liquid hand soap are described in the examples below.

### Example 1

### Dual Chamber Dispenser

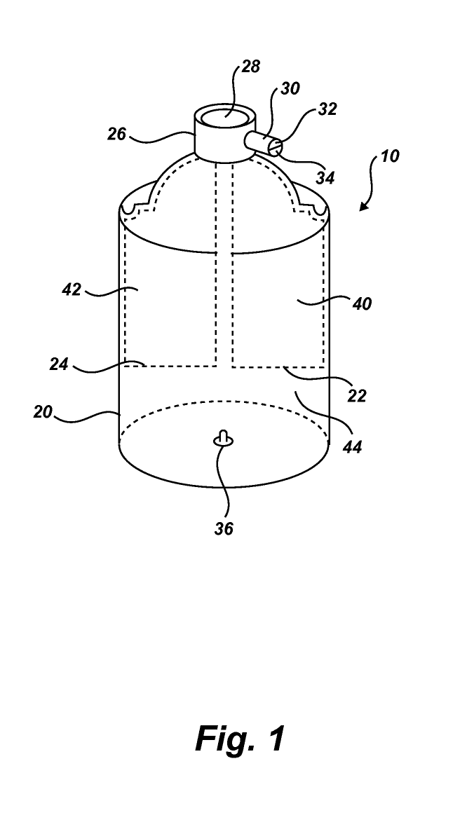

An exemplary combination dispenser is described with reference to FIG. 1. FIG. 1 shows a dual chamber container utilizing a dual bag-in-can design. The dispenser 10 includes the container 20, with valve cap 26. Within the container are two collapsible bags 22 and 24 respectively that contain volumes of shaving gel 40 and liquid hand soap 42. Below the bags is propellant space 44. Propellant is injected into container 20 through bottom, propellant fill port 36, and provides pressure to push the gel and soap out of the container when release valves are actuated. Valve cap 26 includes two internal valve assemblies (not shown) that are controlled by a common trigger 28 releasing both the shaving gel and liquid hand soap through dispensing port 30 through separate channels and apertures 32 and 34. The dispensed combination can then be mixed in the user's hands and applied to the beard area or other skin surface to be shaved.

### Example 2

### Dual Container Dispenser

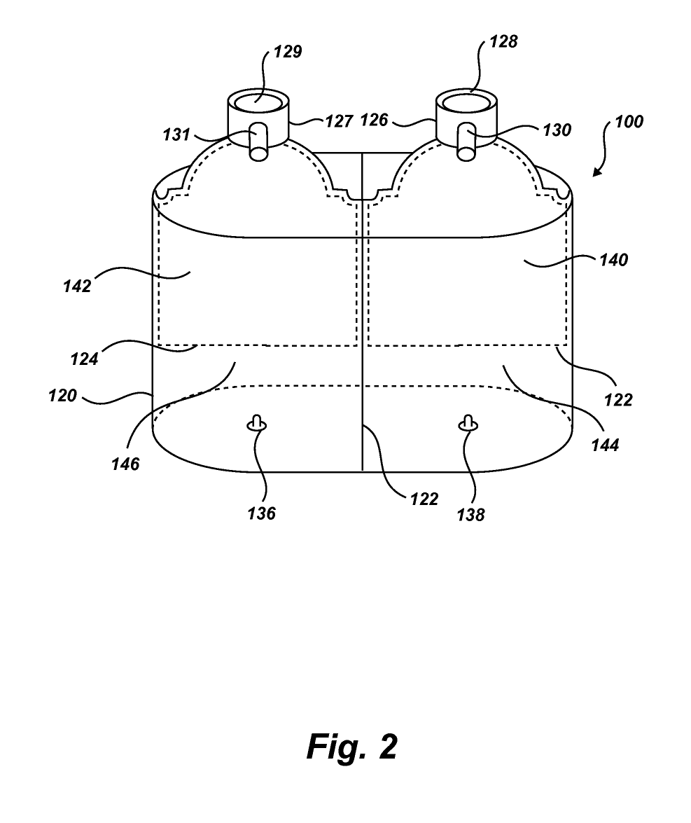

An exemplary dual container dispenser is described with reference to FIG. 2. The dispenser 100 utilizes a bag-in-can design for each chamber. The dispenser includes outer container wall 120 and inner container barrier wall 121 defining 2 separate chambers. Within the chambers are collapsible bags 122 and 124 that contain shaving gel and liquid hand soap respectively. Below the collapsible bags are propellant spaces 144 and 146. Propellant is injected into the propellant spaces through propellant fill ports 136 and 138. Dispensing of the shaving gel and liquid hand soap is controlled using valve caps 126 and 127, which include internal valve assemblies (not shown), valve trigger caps 128 and 129, and outlet spouts 130 and 131.

### Example 3

### Exemplary Shaving Gel and Liquid Hand Soap

An exemplary combination of shaving gel and liquid hand soap is described be reference to previously described shaving gel and liquid had soap formulations.

The shaving gel formulation as described in U.S. Pat. No. 5,560,859 (incorporated herein by reference in its entirety) is prepared from the following ingredients as described below:

| Ingredients | % by wt. |
|---|---|
| Water | 77.95 |
| Palmitic Acid (Hystrene 9016) | 9.74 |
| Triethanolamine | 5.65 |
| Propylene Glycol Isostearate (Emerest 2384) | 1.70 |
| Sorbitol (Liponic 70 NC) | 1.46 |
| PEG-14M (Polyox WSRN-3000) | 0.49 |
| Fragrance | 0.39 |
| Hydroxypropylcellulose (Klucel) | 0.02 |
| Dyes (e.g., FD&C Blue #1 and D&C Yellow #10) | Q.S. |
| Isopentane/Isobutane 85/15 Blend | 2.60 |
| | 100.00% |

The palmitic acid was melted at 75.degree.-80.degree. C. The hydroxypropylcellulose and propylene glycol isostearate were mixed to obtain Mixture 1 which was added to the melted palmitic acid. The Polyox WSRN-3000 was dissolved in substantially all the water to obtain Solution 1. Solution 1 was then warmed to 75.degree.-80 degree C. and maintained at this temperature. The sorbitol and dyes were then added to Solution 1 to form Solution 2. Solution 2 was then added to Mixture 1 to obtain Mixture 2. The 50:50 solution of triethanolamine and water was added to Mixture 2 at 75-80 degree C. to obtain Mixture 3. Mixture 3 was then cooled to 50 degree C. and the fragrance was added to obtain Mixture 4. Mixture 4 was then cooled to 2 degree to 8 degree C. and an 85:15 blend of isopentane/isobutane at a 2.60% level was introduced into Mixture 4.

A liquid hand soap for this exemplary combination is described in U.S. Pat. No. 5,296,158 (incorporated herein by reference in its entirety), prepared as described therein. Additional suitable formulations are also described therein.

The Brookfield viscosity of the following liquid soap is about 30,000 cps. The Iodine Value of the fatty acids therein is about zero and its titer is about 59 degree C. This soap has totals of about 10.2% soap and 6.85% free fatty acid and 2.4% sarcosinate. The soap to free fatty acid (FFA) ratio is about 1:0.67.

| Formula | 1A | 1B |
|---|---|---|
| Ingredients | Wt. % | Wt. % |
| Stearic Acid | 7.55 | 4.53 |
| Palmitic Acid | 6.23 | 3.74 |
| Myristic Acid | 8.72 | 5.23 |
| Lauric Acid | 3.52 | 2.11 |
| Triclosan | 0.30 | 0.18 |
| KOH (87%) | 3.86 | 2.32 |
| Glycerine | 15.00 | 9.00 |
| Mayoquest (45%)* | 0.44 | 0.26 |
| Sodium Lauroyl | 13.33 | 8.00 |
| Sarcosinate (30%) | 0.50 | 0.30 |
| JR-400 | | |
| Aloe Vera Powder | 0.01 | 0.01 |
| Perfume | 0.30 | 0.18 |
| Total Water (approx.) | 50.00 | 70.00 |

*Mayoquest is a 50/50 mixture of HEDP/DPTA

A liquid soap (Formulation 1B) is made by first mixing the ingredients of “1A” as follows:

1. Mix and melt all of the fatty acids with the Triclosan into a jacketed vessel and heat to 80 degree C.

2. Dissolve the KOH pellets with water to make a 38% solution by weight.

3. Mix the glycerine, sodium or potassium lauroyl sarosinate, JR-400, Mayoquest, and water in a separate jacketed vessel and heat to 80 degree C.

4. Transfer the melted fatty acid mix of Step 1 into a vacuum vessel which contains an internal homogenizer, wall scrapers and paddle mixers, e.g., a Mizuho Brand Automatic Driving Type Vacuum Emulsifier, Model APVQ-3DP, sold by Mizuho Industrial Co., Ltd., or a T.K. AGI Homo Mixer Hodel 2M-2, made by Tokushu Kika Kogyo Co., Ltd. While vacuum is not essential, it is highly preferred so that the intermediate product has a specific gravity of about 1±0.05.

5. Slowly add the KOH solution under vacuum of about 400 mm Hg while mixing and homogenizing during saponifying. Maintain temperature controlled to 80±5 degree C. while mixing.

6. After the saponification is complete, add the water mix of Step 3 under vacuum while continuing mixing and homogenizing. Maintain temperature controlled to 80±5 degree C. while mixing to obtain a phase stable melt.

7. Immediately begin cooling from 80 degree C. to 50 degree C. at a 3 degree C./minute rate. Maintain mixing and vacuum during cooling step but stop homogenizing.

8. Dissolve the aloe vera powder in water and add at 50 degree C.

9. Cool from 50 degree C. to 35 degree C. at a 0.5 degree C./minute rate under vacuum and while mixing.

10. At 35 degree C. stop the vacuum and add the perfume. Continue cooling with mixing until final mix reaches about 30 degree C. At 30 degree C., stop cooling and unload the mix from the vessel.

11. The cooled melt of Step 10 (1A) is then diluted with distilled water at about room temperature. The water and the cooled melt is first mixed gently to provide a uniform slurry and then transferred to the vacuum vessel of Step 4 and homogenized for about 10 minutes under about 600 Mm Hg to provide an aqueous (70% water) liquid soap dispersoidal (Formulation 1B).

Formulation 1B can further include stabilizers, such as the combination described in the above-cited patent.

### Example 4

### Exemplary Razors Having Separate Shavers

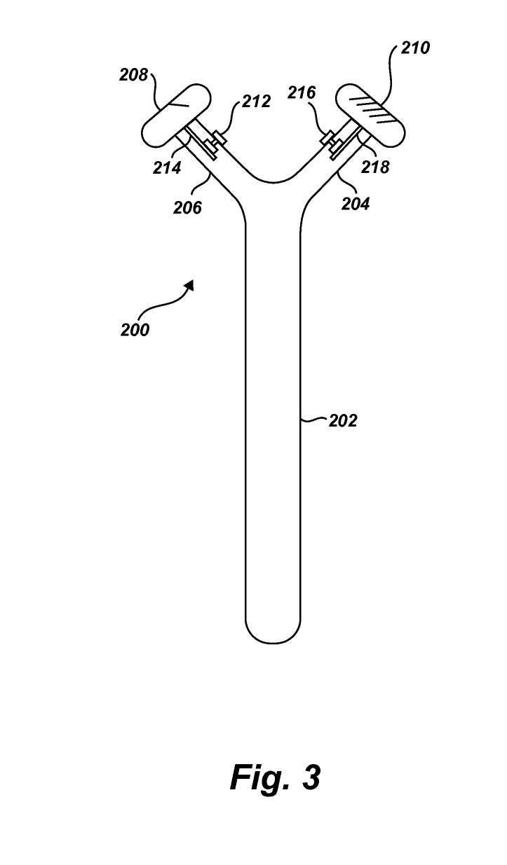

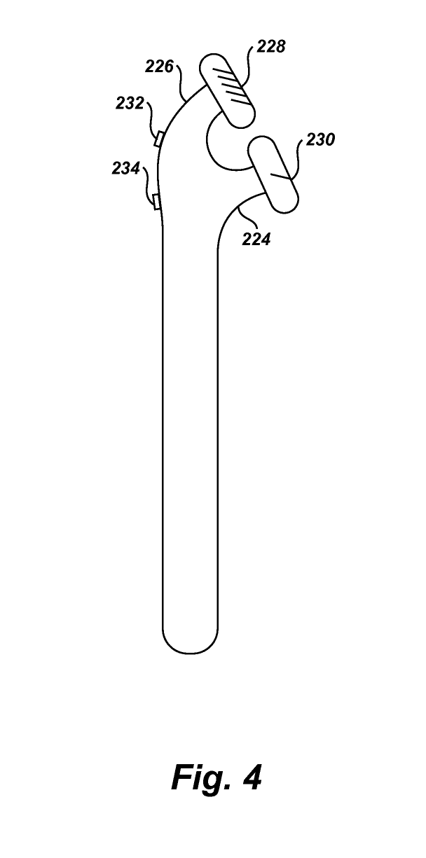

Razors which include separate blade units having differing shaving closeness characteristics are illustrated in FIGS. 3 and 4. As shown in FIG. 3, a dual head razor 200 can be used in which the two heads are positioned approximately symmetrically about the longitudinal axis of the razor handle 202. At one end of the handle are two arms 204 and 206 to which mount the different blade units 208 and 210. In this illustration, blade unit 208 includes a single blade strip, while blade unit 210 includes 5 blade strips. The blade units are retained via retention inserts 214 and 218, which can be released by pressing on release buttons 212 and 216. The blade unit having one blade strip is used to provide an initial shave (e.g., a “rough cut”), followed by a close cut using the 5-blade strip blade unit. Of course, the illustrated blade units can have different numbers of blade strips, e.g., 2 blade strips for the “rough cut” side, and 3, 4, or 5 blade strips for the close cut side.

One alternative design is illustrated in FIG. 4. As illustrated, dual head razor 220 includes handle 222, which, at one end, splits into separate mounting post sections 224 and 226. Mounting post section 224 holds the initial or rough cut blade unit 230, while mounting post section 226 holds the close cut blade unit 228. Similar to the razor shown in FIG. 3, the blade units have 1 and 5 blade strips respectively, but can also be replaced with blade units having different numbers of blades. As illustrated, the skin contact surfaces (i.e., shaving surfaces) of the two different blade units are in the same plane, such that both blade units will function in each stroke. Alternatively, the blade units can be in different planes arranged such that the first blade unit can be used to shave an area, then the handle can be pivoted such that the handle portion is at a steeper angle to the user's skin surface, bringing the second blade unit into contact position and allowing shaving using the second blade unit.

Multi-head razor designs are not limited to dual-head razors, but instead can have additional heads with associated blade units. For example, razors having three or four heads can be provided. As an example, a three-head razor can be designed with the three heads arrayed at angles of about 120 degrees. Similarly, a four-head razor can be designed with the four heads arrayed at angles of about 90 degrees. In most cases it will be preferred that the blade units for such multi-head razors be readily changeable, typically disposable.

In one type of design, the types of dual-head designs above can be combined in a single razor. Thus, such a razor would have at least 3 heads. At least two heads would be arranged in the manner described for FIG. 3 and/or at least two heads would be arranged in the manner described for FIG. 4. In most cases, such razors would have 3 or 4 heads.

### Example 5

### Exemplary Razors Having Improved Cleaning

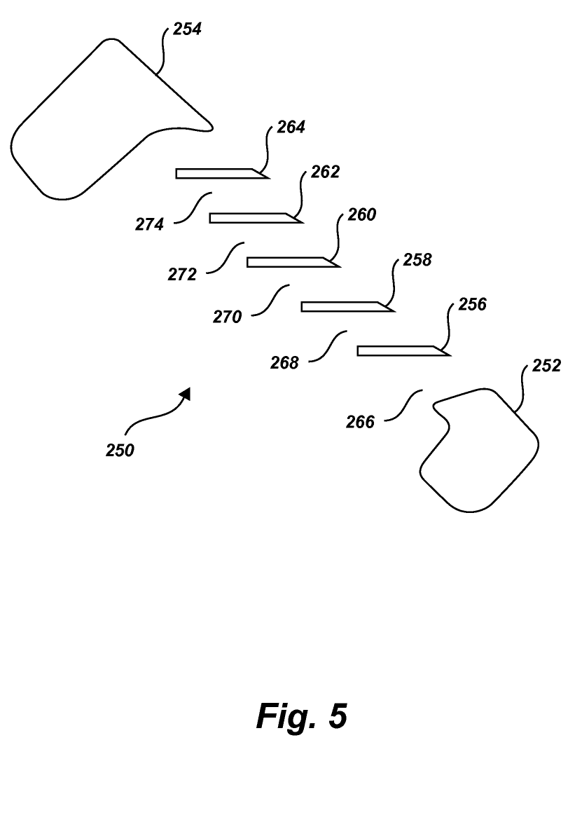

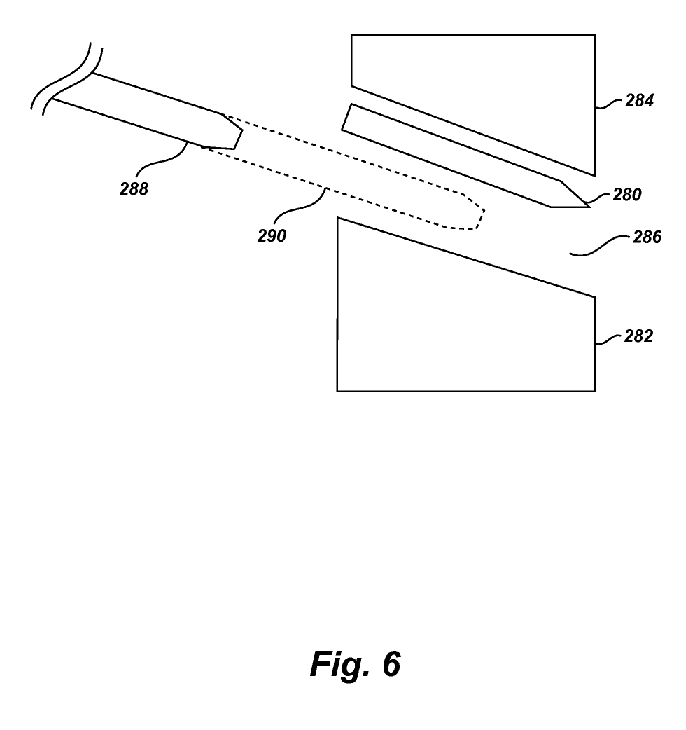

Razor blade units which are easier to clean as compared to present multi-blade blade strip blade units are illustrated in FIGS. 5 and 6. FIG. 5 shows a cross-section through the approximate middle of a blade unit 250. The blade unit has outer support frame members 252 (leading edge) and 254 (trailing edge) which form part of the support structure of the blade unit and also provide bearing surfaces controlling contact with the user's skin. Blade strips 256, 258, 260, 262 and 264 are mounted at their ends to the side portions of the support structure (not shown). Preceding the respective blade strips are debris throats 266, 268, 279, 272, and 274 respectively. As illustrated, the first debris throat 266 is relatively wide, allowing large amounts of shaved hair debris to pass through the throat without clogging and/or making removal of debris using water easier. Similarly, the second debris throat 268 is also relatively wide, and thus also allowing large amounts of cut hair debris to pass through the throat and/or assisting in clearing accumulated debris. The third debris throat 270 is intermediate in width and thus has intermediate debris capacity, while the fourth and fifth debris throats 272 and 274 respectively are narrower and therefore have the least debris capacity. In normal use, the wider debris throat(s) at the leading edge will be subjected to the greatest amount of debris because most of the cut is accomplished with the leading blade strips. On the other hand, the trailing debris throats will be subjected to much less debris because the previous blade strips will have cut most of the hairs. Consequently, the latter blade strips can be placed closer together to provide a closer shave without compromising debris clearing ability.

In one variant of this general design, an additional surface which bears on the skin can be included following the first blade strip or first pair of blade strips. Inclusion of such skin-bearing surface may increase safety and comfort during use of the blade unit. Similar skin-bearing surfaces are commonly included in current shaving blade units, and can be readily adapted for this purpose. The spacing of this skin-bearing surface from the next blade strip then defines the debris throat for that next blade strip.

Another feature which can assist debris clearing in either conventional multi-blade strip blade units or in blade units of the general type shown in FIG. 5 is illustrated in FIG. 6, and includes a clearing plunger. For simplicity, a plunger with a blade unit having only one blade strip is shown, but the system can likewise be used with blade units having multiple blade strips where the clearing plunger clears one or more of the debris throats. As illustrated, a blade strip 280 is mounted in a blade unit, bracketed by front and rear support frame members 282 and 284 respectively. Preceding blade strip 280 is debris throat 286. Following accumulation (e.g., packing) of debris in debris throat 286, plunger 288 can be extended into position 290 (shown with dashed lines) into (or even through) the debris throat thereby pushing out accumulated debris. The plunger will typically be actuated by using a button, slide, or the like (not shown) on the corresponding razor handle linked with the plunger.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the shaving gel and liquid hand soap formulations. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention.

Thus, additional embodiments are within the scope of the invention and within the following claims.

1. A shaving composition, comprising
20-80% by weight of a shaving gel, shaving foam, or shaving cream; and
20-80% by weight of a liquid hand soap.

2-5. (canceled)

6. A shaving composition dispenser comprising
two chambers, wherein the first chamber contains a shaving gel, shaving foam, or shaving cream, the second chamber contains a liquid hand soap, and said dispenser dispenses a pre-selected ratio of each of said liquid and said shaving gel, shaving foam, or shaving cream.

7-12. (canceled)

13. A method for shaving hair from a skin surface, comprising
applying a shaving gel, foam, or cream and a liquid hand or body soap to a skin surface to be shaved; and
shaving said skin surface.

14-16. (canceled)

17. A method for shaving hair from a skin surface, comprising
shaving said skin surface using a razor having a first blade unit comprising at least 1 blade strip followed by shaving said skin surface with a second blade unit comprising at least one more blade strip than is contained in said first blade unit.

18. The method of claim 17, wherein said skin surface is washed with a liquid hand or body soap immediately prior to said shaving.

19. The method of claim 17, wherein a combination liquid hand or body soap and shaving cream or gel or foam is used for lubrication and softening for said shaving.

20. The method of claim 17, wherein at least two different razors are used.

21. The method of claim 17, wherein a multi-head razor is used.

22. The method of claim 21, wherein said multi-head razor comprises at least two heads in sequential orientation.

23. The method of claim 22, wherein said multi-head razor comprises at least two blade units in the same plane.

24. The method of claim 22, wherein blade units on said at least two heads are arranged such that, beginning from a orientation in which a blade unit is in proper orientation for shaving, changing the angle of the razor handle to a steeper angle relative to the user's skin brings a different blade unit into contact orientation.

25. The method of claim 22, wherein blade units on said at least two heads are configured such that changing from one head unit to another requires rotating said razor about the longitudinal axis of the razor handle by approximately 90, 120, or 80 degrees.

26. A shaving kit, comprising
at least two substantially different types of blade units, wherein said blade units are effective for producing at least two substantially different shaving closeness levels or are of substantially different cost or both, packaged together.

27. The shaving kit of claim 26, further comprising instructions for using said substantially different types of blade units jointly in a shaving process.

28. The shaving kit of claim 26, further comprising a multi-head razor handle which simultaneously mounts blade units of each of said at least two substantially different types of blade units.

29. The method of claim 26, wherein said instructions comprise directions for shaving using a combination of a liquid hand or body soap and a shaving gel, foam, or cream.

30. The method of claim 26, wherein said instructions comprise directions for shaving using sequential application of a liquid hand or body soap followed by application of a shaving gel, foam, or cream.

31. The method of claim 26, wherein said instructions comprise directions for shaving using sequential application of a shaving gel, foam, or cream followed by application of a liquid hand or body soap.

32. A multi-head razor comprising at least two substantially different blade units which respectively have different numbers of blade strips.

33. A razor blade unit comprising
at least three blade strips having decreasing spacing, wherein the spacing between the first and second blade strips is greater than between the next-to-last and last blade strips.

34. The razor blade unit of claim 33, further comprising a debris throat preceding the first blade strip and between each pair of blade strips, wherein the debris throat before the first blade strip is at least 50% wider than the debris throat between the next to last and last blade strips.

35. The razor blade unit of claim 33, wherein said razor blade unit is mounted on a razor handle and said handle comprises a clearing plunger operable using a contact on the razor handle such that said clearing plunger extends into or through a least one debris throat.
